(19) United States
(12) Patent Application Publication
(10) Pub. No.: US 2025/0356997 A1
(43) Pub. Date: Nov. 20, 2025

(54) SYSTEMS AND METHODS TO VISUALIZE ALERT COUNTS TO IDENTIFY COMMUNICATION ISSUES PROMPTLY

(71) Applicant: KONINKLIJKE PHILIPS N.V., EINDHOVEN (NL)

(72) Inventors: Johannes Henricus Maria KORST, EINDHOVEN (NL); Serverius Petrus Paulus PRONK, VUGHT (NL); Rithesh SREENIVASAN, BENGALURU (IN)

(21) Appl. No.: 18/871,203

(22) PCT Filed: Jun. 5, 2023
(86) PCT No.: PCT/EP2023/064935

§ 371 Date: Dec. 3, 2024

Related U.S. Application Data
(60) Provisional application No. 63/352,311, filed on Jun. 15, 2022.

(51) Int. Cl. G16H 40/40; G16H 30/20

(52) U.S. Cl. CPC G16H 40/40; G16H 30/20

(57) ABSTRACT

An alert monitoring method (100) includes receiving alerts (34) generated by n medical databases (12) servicing respective medical entities, each alert being timestamped with a receipt time when the alert was received at the electronic processor and each alert being associated to the medical database that generated the alert; generating a grid representation (36) of the alerts over a time span, the grid representation comprising a grid of blocks; generating a heat map (38) representing the alerts over the time span and having blocks corresponding to the blocks indexed of the grid representation by mapping the values of the respective blocks of the grid representation to colors using a color scale; and displaying the heat map on a color display (24) that is operatively connected with the electronic processor.

### FIELD

The following relates generally to the radiology arts, electronic database maintenance arts, radiology database maintenance arts, picture archiving and communication system (PACS) maintenance arts, and related arts.

### BACKGROUND

The medical images that are produced by medical imaging systems, such as magnetic resonance (MR), computed tomography (CT), and image guided therapy (IGT) systems, are stored for later viewing in a secure storage system commonly called a Picture Archiving and Communication System (PACS). Radiologists access and interpret the images from a PACS to report their findings in a so-called imaging or radiology report. If images are not accessible because a PACS is down, or if loading images from a PACS is slow due to a degraded performance, then radiologists are not able to do their work efficiently. Also the work of other users of a PACS, such as biomedical engineers (“bio-meds”), technicians and referring physicians, will be adversely affected by a degraded performance of a PACS. Consequently, it should be avoided that a PACS experiences a reduced functionality, whenever possible. Nowadays, a PACS is built using standard computer technology, where often virtual machine technology is used to achieve hardware platform independency and to allow easy redirecting of virtual processing nodes when the underlying hardware platform experiences problems.

To proactively avoid unplanned down time of a medical database, such as a PACS, a monitoring system can repeatedly check specific parameter values (such as central processing unit (CPU), memory and disk usage as well as queue lengths) for the various nodes present in a PACS. The monitoring system can generate an alert whenever a parameter value exceeds a WARNING or CRITICAL threshold. These alerts are sent to a central location and presented on a dashboard that is used by a proactive monitoring team. Using dedicated business rules, the proactive monitoring team members can use these alerts to create tickets/cases that are next handled by remote service engineers.

The alerts can be characterized by the site and device at which they are generated, as well as by an alert type specifying the type of parameter (CPU, memory, disk, etc.). In addition, an alert will typically contain additional information on the specific hardware parameter that is monitored as well as the parameter values that are observed. A site may be a small hospital or a large hospital or even a large hospital chain consisting of multiple locations.

The average number of alerts that are generated per site will depend on the size of the site. Furthermore, for a given site, the number of alerts generated per time unit may vary over time, depending on the usage of the devices at the site and the number of issues that can cause alerts to be generated.

The alerts generated at the various sites are sent to a central server, often using different communication mechanisms, depending on for example, whether the alerts are sent over a dedicated communication channel or over a series of multiple communication networks that are shared by various other applications.

Due to various reasons, it may happen that alerts are lost or delayed in the communication from the specific site to the central storage server. For example, as the communication networks are typically not designed for worst-case situations, there will occasionally be more alerts generated per time unit than can be transmitted immediately. Hence, some alerts may be locally stored for later transmission and consequently arrive at the central server with additional delays, or given the limited local storage capacity, they may even never arrive at the central server.

In addition, there can be various other reasons why alerts may not arrive at the central server, or arrive only with additional delays. Firewall issues and unexpected side effects of changes in the configurations of the various involved parts may also results in delayed or missing data.

Depending on where the communication problems appear, the delayed or missing data may correspond to only one site, a number of sites located in the same region, or may even correspond to all sites when the communication problem appears in the last step of a series of communication steps.

To correctly interpret the presence or absence of alerts, it is important that such communication issues are detected as early as possible. Given that the number of generated alerts may vary considerably over time, it is not trivial to identify communication issues in the presence of this high variability.

The following discloses certain improvements to overcome these problems and others.

### SUMMARY

In some embodiments disclosed herein, a non-transitory computer readable medium stores instructions readable and executable by an electronic processor to perform an alert monitoring method including receiving, at the electronic processor and via an electronic network, alerts generated by n medical databases servicing respective medical entities, each alert being timestamped with a receipt time when the alert was received at the electronic processor and each alert being associated to the medical database that generated the alert; generating a grid representation of the alerts over a time span T, the grid representation comprising a grid of blocks indexed (i, j) where index i=1, . . . , n indexes the n medical facilities and index j=1, . . . , k indexes k time-ordered time intervals t₁, . . . , tₖ spanning the time span T where the time intervals t₁, . . . , tₖ are of equal time duration, wherein each block (i, j) of the grid has a value a_{i,j} indicative of a number of alerts received from medical database i over the time interval tⱼ; generating a heat map representing the alerts over the time span T and having blocks corresponding to the blocks indexed (i, j) of the grid representation by mapping the values a_{i,j} of the respective blocks of the grid representation to colors using a color scale; and displaying the heat map on a color display that is operatively connected with the electronic processor.

In some embodiments disclosed herein, an alert monitoring apparatus includes a server computer programmed to receive alerts generated by n medical databases servicing respective medical entities, each alert being timestamped with a receipt time when the alert was received at the electronic processor and each alert being associated to the medical database that generated the alert; generate a grid representation of the alerts over a time span T, the grid representation comprising a grid of blocks indexed (i, j) where index i=1, . . . , n indexes the n medical facilities and index j=1, . . . , k indexes k time-ordered time intervals t₁, . . . , tₖ spanning the time span T where the time intervals t₁, . . . , tₖ are of equal time duration, wherein each block (i, j) of the grid has a value a_{i,j} indicative of a number of alerts received from medical database i over the time interval tⱼ, wherein the mapping comprises constructing n medical database-specific color scales C₁, . . . , Cₙ corresponding to the n medical databases, the color scale Cᵢ for corresponding medical database i being scaled based on the values a_{i,1}, . . . , a_{i,k} for the alerts received from the medical database i; and mapping the values a_{i,1}, . . . , aₖ for each respective medical database i to colors of the database-specific color scale Cᵢ constructed for the medical database i; wherein the constructing of each database-specific color scale Cᵢ includes scaling by a median absolute difference of the values a_{i,1}, . . . , a_{i,k} for the alerts received from the medical database i; generate a heat map representing the alerts over the time span T and having blocks corresponding to the blocks indexed (i, j) of the grid representation by mapping the values a_{i,j} of the respective blocks of the grid representation to colors using a color scale; and display the heat map on a color display that is operatively connected with the electronic processor.

In some embodiments disclosed herein, an alert monitoring method includes receiving, at an electronic processor and via an electronic network, alerts generated by n medical databases servicing respective medical entities, each alert being timestamped with a receipt time when the alert was received at the electronic processor and each alert being associated to the medical database that generated the alert; generating a grid representation of the alerts over a time span T, the grid representation comprising a grid of blocks indexed (i, j) where index i=1, . . . , n indexes the n medical facilities and index j=1, . . . , k indexes k time-ordered time intervals t₁, . . . , tₖ spanning the time span T where the time intervals t₁, . . . , tₖ are of equal time duration, wherein each block (i, j) of the grid has a value a_{i,j} indicative of a number of alerts received from medical database i over the time interval tⱼ, wherein the mapping comprises constructing n medical database-specific color scales C₁, . . . , Cₙ corresponding to the n medical databases, the color scale Cᵢ for corresponding medical database i being scaled based on the values a_{i,1}, . . . , a_{i,k} for the alerts received from the medical database i; and mapping the values a_{i,1}, . . . , aₖ for each respective medical database i to colors of the database-specific color scale Cᵢ constructed for the medical database i; wherein the constructing of each database-specific color scale Cᵢ includes scaling by a median absolute difference of the values a_{i,1}, . . . , a_{i,k} for the alerts received from the medical database i; wherein the constructing of each database-specific color scale Cᵢ includes scaling by a median absolute difference median (|x−μ|) where μ is the median value of the set {a_{i,1}, . . . , a_{i,k}}; generating a heat map representing the alerts over the time span T and having blocks corresponding to the blocks indexed (i, j) of the grid representation by mapping the values a_{i,j} of the respective blocks of the grid representation to colors using a color scale; and displaying the heat map on a color display that is operatively connected with the electronic processor.

One advantage resides in visualizing a number of alerts per time unit per site for various medical sites.

Another advantage resides in providing a visualization effective for recognizing lost or delayed alerts.

Another advantage resides in providing a visualization effective for recognizing communication issues for transfer of alerts from local sites to a central server.

Another advantage resides in providing a visualization of alert counts that highlights communication gaps.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an alert monitoring apparatus in accordance with the present disclosure.

FIG. 2 diagrammatically illustrates an alert monitoring method using the apparatus of FIG. 1.

FIG. 3 shows a heat map generated by the apparatus of FIG. 1.

### DETAILED DESCRIPTION

A vendor can provide a Picture Archiving and Communication System (PACS) product to many medical service providers. At each hospital (or, more generally, medical entity), the PACS is implemented as a local server, either running directly on a physical server computer or running in a virtual machine (VM) on a physical server computer, which may be supplied to the hospital by the vendor, or could be a hospital-owned server. The medical images and associated data (e.g., radiology reports) are stored at this local server, which beneficially facilitates efficient transfer of the large image files.

Such vendors can also provide a service component to the PACS product. In this service component, a central server at the vendor connects with the local hospital servers at various time intervals, and receives status information pertaining to the local PACS server including alerts, which constitute information about hardware or software issues that adversely (or may adversely) impact operation of the local PACS server. For example, an alert may be issued if a PACS component or operational aspect such as central processing unit (CPU), network bandwidth, storage, database, or so forth is becoming overloaded. Some alerts are primarily informational (e.g., CPU usage above 80%), while others may be urgent (e.g., loss of network connectivity). At the vendor, engineers monitor these alerts and take remedial actions such as remotely updating resources of the VM or scheduling an on-site visit to take action locally.

A problem arises in that the number of alerts is large, so that it may be difficult for engineers to prioritize remediation of alerts. Furthermore, for various reasons there can be latency in receiving alerts, so that engineers may be misled into believing a particular local PACS database is running smoothly when in fact it is out of communication.

To address these problems, the following discloses a heat map representation of the number of alerts per unit time as a function of node (i.e. hospital/PACS server) and time. The disclosed heat mapping approaches address some difficulties recognized herein in obtaining a suitable heat map representation. These include difficulties in scaling the alert counts across time and different hospitals. For example, if all hospitals exhibit a range of 0-100 alerts/day and the color coding of the heat map uses green for zero, red for 100, and various shades ranging from green through yellow to red for intermediate values, then it may be the case that a large hospital will always be in the red (more than 100 alerts per day) while a small hospital may always be green (only a handful of alerts per day). Yet, a quantity such as 8 alerts per day for a small hospital that has only a few dozen PACS database retrievals per day could indicate a serious problem; whereas more than 100 alerts per day for a large hospital PACS against which there are many thousands of database retrievals per day may be acceptable. To address this, per-hospital scaling is used in the disclosed embodiments. Furthermore, it was found that scaling using median value improved stability of the heat map. Finally, since a value of zero alerts per unit time is of special importance as it often indicates loss of connectivity with the hospital PACS, a special color can be designated for zero.

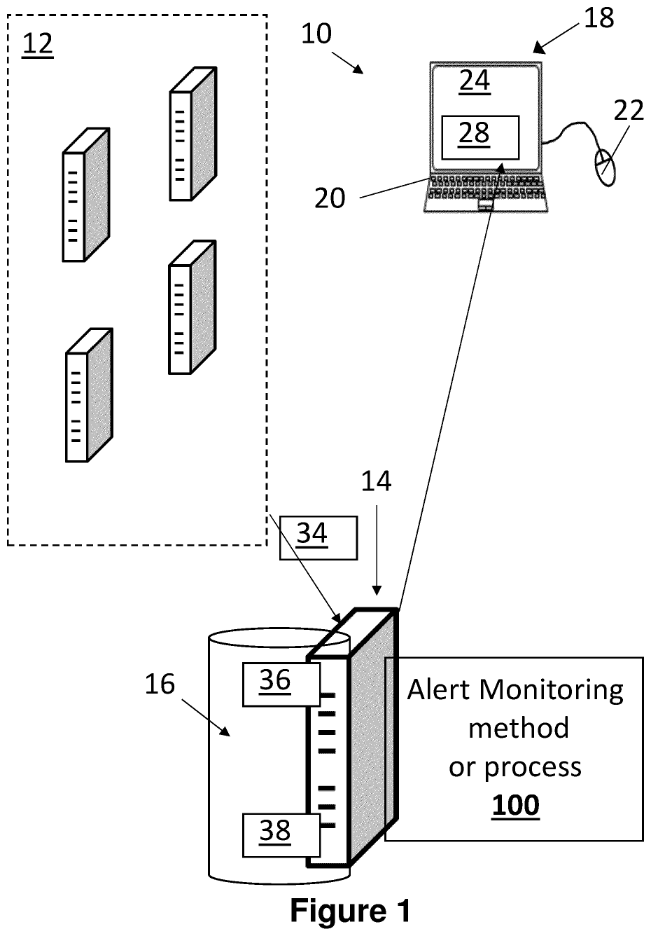

With reference to FIG. 1, an illustrative apparatus 10 for monitoring alerts from one or more associated databases 12 is shown. The databases 12 can comprise n medical databases 12 servicing respective medical entities. While four such databases 12 are shown in FIG. 1, any suitable number of n medical databases 12 can be included. In some embodiments, the n medical databases 12 comprise n respective picture archiving and communication system (PACS) databases, although this is merely an example and the medical databases 12 may in general be assigned other nomenclatures. For example, a medical database servicing a medical entity which is a cardiology department might be a cardiovascular information system (CVIS). As another example, a medical database servicing a medical entity might be an integrated PACS/RIS system where “RIS” refers to “Radiology Information System”. These are merely further examples. The medical databases 12 can comprise, for example, a medical informatic system, such as PACS, RIS, etc. Each PACS or other medical database 12 typically comprises a server computer running suitable software implementing the PACS, either running directly on the server (i.e. running on silicon) or running in a virtual machine (VM) that runs on the server. By way of nonlimiting illustrative example, a medical database 12 could comprise a server computer running the IntelliSpace PACS (available from Koninklijke Philips N. V., Eindhoven, the Netherlands).

As shown in FIG. 1, the monitoring apparatus 10 includes, or is accessible by, a server computer 14 typically disposed remotely from the databases 12. The server computer 14 comprises a computer or other programmable electronic device that includes or has operative access to a non-transitory computer readable medium. The server computer 14 is generally separate from any of the server computers implementing the n medical databases 12, and the server computer 14 typically communicates with the n medical databases 12 via the Internet, optionally along with one or more local area networks (LAN) or wide-area networks (WAN), such as a LAN or WAN of the hospital at which the PACS 12 is located, and/or a LAN or WAN of the vendor or of a cloud computing service provider providing the server computer 14 to the vendor. It will be appreciated that the server computer 14 could be implemented as a plurality of server computers, e.g., interconnected to form a server cluster, cloud computing resource, or so forth, to perform more complex computational tasks.

An electronic processing device 18, such as a workstation computer, or more generally a computer, a mobile device (e.g., a tablet computer), is operable by a service engineer (SE), information technology (IT) professional, or the like to provide a user interface with an alert monitoring method or process 100 running on the server computer 14. The electronic processing device 18 includes typical components for a user interfacing computer, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices. To display a heat map the display device 24 should preferably be a color display device.

The non-transitory storage media 16 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non- transitory medium or media 16 herein is to be broadly construed as encompassing a single medium or multiple media of the same or different types. Likewise, the electronic processor 20 may be embodied as a single electronic processor or as two or more electronic processors. The non- transitory storage media 16 stores instructions executable by the at least one electronic processor 20. The instructions include instructions to generate a visualization of a graphical user interface (GUI) 28 for display on the display device 24.

The databases 12 communicate with the server computer 14 via a communication link, which typically comprises an electronic network including the Internet augmented by local area networks (e.g. LAN or WAN) for electronic data communications. The electronic processing device 18 may be a dumb terminal connected with the server computer 14 via a LAN, WAN, Internet, or so forth. In the illustrative example, the server computer 14 handles the communication with the n medical databases 12 and the computation of the heat map representing alert counts as disclosed herein, and the electronic processing device 18 primarily serves as the user interfacing device including displaying the heat map on the display 24. However, this is merely an illustrative example, and the processing may be variously distributed between the server 14 and user interfacing processing device 18. In some embodiments, only one computer may be provided which includes all functionality-e.g., the server computer 14 could be omitted and its functionality performed solely by the single user interfacing computer 18.

The apparatus 10 is configured as described above to perform an alert monitoring method or process 100 of monitoring alerts 34 received from the database(s) 12. The database 16 stores instructions executable by the server computer 14 (and/or by the electronic processing device 18) to perform the alert monitoring method or process 100 which includes presenting a heat map 38 representing the alerts over a time span. In some examples, the method 100 may be performed at least in part by cloud processing (that is, the server computer 16 may be implemented as a cloud computing resource comprising an ad hoc network of server computers).

To proactively avoid unplanned down time of the databases 12, the monitoring apparatus 10 can repeatedly check specific parameter values (such as CPU, memory disk usage, queue lengths, and so forth) for the various nodes (i.e., medical devices) present in the databases 12. The monitoring apparatus 10 can generate an alert 34 if a value of a performance parameter for the database 12 meets a specified criterion, such as whenever a parameter value exceeds a predetermined threshold. These alerts 34 are sent to the server computer 14 and/or the electronic processing device 18, and presented on the GUI 28. A user can use these alerts 34 to create tickets/cases that are next handled by remote service engineers.

The alerts 34 can be characterized by the location and device at which they are generated, as well as by an alert type specifying the type of parameter (CPU, memory, disk, etc.). In addition, the alert 34 can typically contain additional information on the specific hardware parameter that is monitored as well as the parameter values that are observed. A site may be a small hospital or a large hospital or even a large hospital chain consisting of multiple locations.

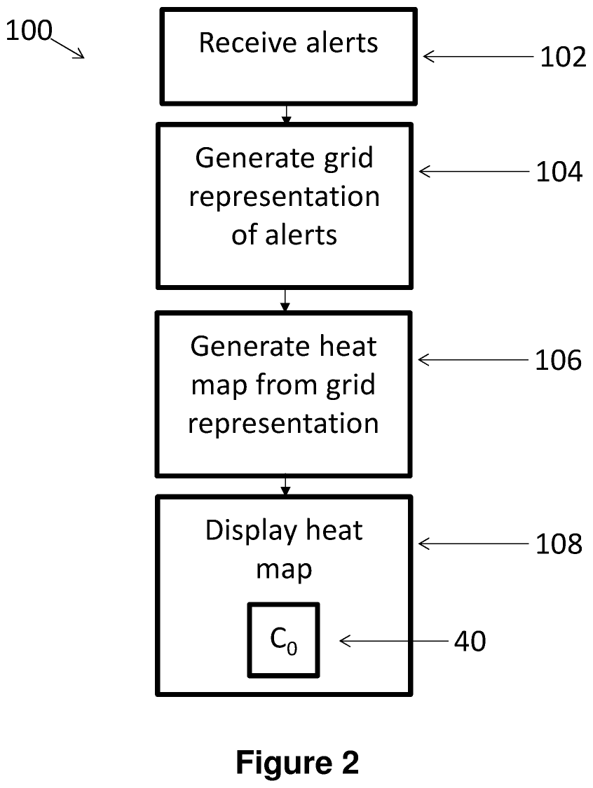

With reference to FIG. 2, and with continuing reference to FIG. 1, an illustrative embodiment of an instance of the alert monitoring method 100 is diagrammatically shown as a flowchart.

At an operation 102, alerts 34 generated by the medical databases 12 are received at the server computer 14 via an electronic network (i.e., the Internet). Each alert 34 is timestamped with a receipt time when the alert 34 was received at the server computer 14, and each alert 34 is associated to the medical database 12 that generated the alert 34.

At an operation 104, a grid representation 36 of the alerts over a time span T are generated. The grid representation 36 comprises a grid of blocks indexed (i, j) where index i=1, . . . , n indexes the n medical facilities and index j=1, . . . , k indexes k time-ordered time intervals t₁ . . . , tₖ spanning the time span T where the time intervals t₁, . . . , tₖ are of equal time duration. Each block (i, j) of the grid representation 36 has a value a_{i,j} indicative of a number of alerts 34 received from medical database i over the time interval tⱼ. By way of nonlimiting illustrative example, each time interval tⱼ could be one day, and if the time span T is 30 days, then there are k=30 time-ordered time intervals t₁, . . . , t₃₀ spanning those 30 days.

At an operation 106, a heat map 38 representing the alerts 34 over the time span T and having blocks corresponding to the blocks indexed (i, j) of the grid representation 36 is generated by mapping the values di, j of the respective blocks of the grid representation 36 to colors using a color scale.

In some embodiments, the color scale includes a first color (i.e., red), a second color (i.e., yellow), and a third color (i.e., green). The mapping operation 106 includes mapping to the first color values a_{i,j} that are indicative of a high number of alerts 34 received from medical database i over the time interval t; (i.e., red color values); mapping to the second color values a_{i,j} that are indicative of an intermediate number of alerts 34 received from medical database i over the time interval tⱼ (i.e., yellow color values); and mapping to the third color values a_{i,j} that are indicative of a low number of alerts 34 received from medical database i over the time interval tⱼ (i.e., green color values). In some examples, the color scale further includes a fourth color (i.e., white), and the mapping operation 106 includes mapping to the fourth color values a_{i,j} that are equal to zero. This is merely an illustrative example, and the number of colors in the color scale could be different. Moreover, while the illustrative color scale is described in terms of values of hue (e.g., red, green, yellow, magenta, et cetera) it is to be understood that the color scale can include scaling on the basis of various color properties or combinations of properties, including hue, tint, brightness, shade, saturation, or so forth.

In some embodiments, the mapping operation 106 includes constructing n medical database-specific color scales C₁, . . . , Cₙ corresponding to the n medical databases 12. The color scale Cᵢ for corresponding medical database i are scaled based on the values a_{i,1}, . . . , a_{i,k} for the alerts 34 received from the medical database i. The values a_{i,1}, . . . , aₖ for each respective medical database 12 are mapped to colors of the database-specific color scale Cᵢ constructed for the medical database i. For example, the constructing of each database-specific color scale Cᵢ includes scaling by a median absolute difference of the values a_{i,1}, . . . , a_{i,k} for the alerts 34 received from the medical database i. In another example, the constructing of each database-specific color scale Cᵢ includes scaling by a median absolute difference median_{j=1,k} (|a_{i,j}−μᵢ|) where μᵢ is the median value of the set {a_{i,1}, . . . , a_{i,k}}. As will be further discussed, in some embodiments the color scale includes a special dedicated color, denoted herein as C₀, for zero counts, which is typically expected to occur only (or at least most often) in the case of a loss or delay in communication of the alerts for some time interval.

At an operation 108, the heat map 38 is displayed on a color display (i.e., on the GUI 28 of the display device 24). In some embodiments, a user can select one (or more) block (i, j) of the displayed heat map 38 via the at least one user input device 22. This input is received at the server computer 14, and a list of the alerts 34 received from the medical database 12 indexed i over the time interval tⱼ (i.e., the selected block) is displayed on the display device 24.

In some embodiments, the heat map 38 can be used to predict a potential failure of one or more of the databases 12. For example, if a number of alerts 34 generated by one of the databases 12 is high is relatively short time interval as shown in the heat map 38, then that database 12 may be at risk of malfunction or failure. In another example, the heat map 38 can be used to predict a trend in the number of alerts 34 over time for a particular database 12.

### EXAMPLE

The following describes the apparatus 10 and the method 100 in more detail. The apparatus 10 is configured to visualize the number of alerts per time unit per site for the various sites in one overall dashboard, by using a site-dependent transformation.

By applying this transformation, both issues that are local to a given site as well as issues that involve a large portion of the sites can be identified, taking into account that the different sites may vary considerably in size, i.e., in the number and usage of the devices of a given site.

Let the set of n sites be numbered by 1,2, . . . , n and let time be partitioned in successive non-overlapping time intervals numbered . . . , −1, 0, 1, 2, . . . . Furthermore, let aᵢⱼ ∈ [symbol] denote the number of alerts for site i at time interval j.

In a first embodiment, a state-of-the-art visualization of the number of alerts per time units using a heatmap is disclosed. Assuming that the alert counts per site for k successive time intervals is to be visualized, say for intervals 1, 2, 3, . . . , k, then a n×k matrix of small rectangles and color rectangle r_{i,j} can be used, i.e., the rectangle in the i-th row and the j-th column with a color that depends on alert count a_{i,j}. This can be done as follows. Let aₘᵢₙ=min{a_{i,j}|1<i<n, 1<j<k} and aₘₐₓ=max{a_{i,j}|1<i<n, 1<j<k}. We can now color r_{i,j} using, for example, a color range from green, via yellow to red, where aₘᵢₙ is mapped to green and aₘₐₓ is mapped to red and all intermediate alert counts are mapped appropriately to obtain a continuous transition from green to red, via yellow when a_{i,j} increases from aₘᵢₙ to aₘₐₓ. To obtain a uniform appearance, one can choose all colors from the given range to have the same brightness.

In this embodiment, the visualization embodiment has an important drawback. It does not take into account that the number of alerts for one (large) site may be considerably larger than for another (small) site. As a result, the successive alert counts for the small site will all appear almost identically green, while the successive alert counts for the large site will all appear almost identically red, so that relatively large changes in the alert count of a single site get unnoticed.

To avoid this, a second embodiment of the visualization will use minima and maxima that are specific for the specific site. Let a_{i,min}=min{a_{i,j}|1 ≤j≤k} and a_{i,max}=max{a_{i,j}|1≤j≤k}. For a given site i, r_{i,j} is colored such that a_{i,min} is mapped on green, a_{i,max} is mapped on red, and all intermediate alert counts are mapped appropriately to obtain a continuous transition from green to red, via yellow when a_{i,j} increases from a_{i,min} to a_{i,max}.

As new data arrives, the visualization should be updated, by either extending the matrix with another column, or by removing the first column and by adding an additional last column to keep the number of columns constant. In both cases, the values of a_{i,min} and a_{i,max} may have changed as j now ranges over 1≤j≤k+1 or over 2≤j≤k+1. It may happen that for a number of sites the minimum and maximum values are changed considerably, resulting in considerable changes in the successive visualizations, which is clearly not desired. If for a given site i, a_{i,k+1}>>max{a_{i,j}|1≤j≤k}, then this will result in a transition in color for almost all of the rectangles in the respective i-th row.

To avoid this, the alert counts are mapped in the i-th row onto the color range from green to red, by using the median u and the median absolute difference (MAD) δ of the alert counts in {a_{i,j}|1≤j≤k}. The median absolute difference δ for a given set {a_{i,j}|1≤j≤k} is defined as median (|x−μ|) where u is defined as the median of the set {a_{i,j}|1≤j≤k}.

This can be done as follows. Let any alert count at most μ−c·δ be mapped on green, let alert count equal to u be mapped on yellow, and let any alert count at least μ+c·δ & be mapped on red, for an appropriately chosen value of c, such as for example the value 2. In a similar fashion as in the previous two visualizations, the alert counts between μ−c·δ and μ can be mapped appropriately to obtain a continuous transition from green to yellow, and the alert counts between μ and μ+c·δ. As a result, the visualization will be less influenced by outliers both at the minimum and at the maximum range. By using median and median absolute difference, instead of mean and standard deviation, parameters that are less influenced by outliers are used. If a mean and standard deviation are used, then a single high outlier a_{i,j} would result in mapping the other alert count to colors that are close to the middle color yellow, resulting in seeing hardly any differences between these other alert counts.

The increased stability that was obtained by using the median and median absolute deviation comes at a price, however. The difference between alert counts ranging from 0 to μ−c·δ can no longer be seen, as they are all mapped to the color green. For the application at hand it is beneficial to distinguish between receiving some alerts from a given site and receiving zero alerts from the site, since receiving zero alerts is likely to be due to a loss of communication or a delay in transmitting alerts. Especially, if at rare occasions, it happens that no alerts are received from any of the sites in a given time interval, this should be seen clearly as this points to some communication issue in the communication chain very near the central server.

To clearly see the difference between receiving an amount of alerts that is at most μ−c·δ and receiving zero alerts, an alert count is mapped to a different color 40, denoted herein as color C₀, that clearly stands out of the other colors. For this we can, for example, also choose the color C₀ to be green, but at a much lower brightness (and/or a different hue or other color characteristic) than all the other used colors.

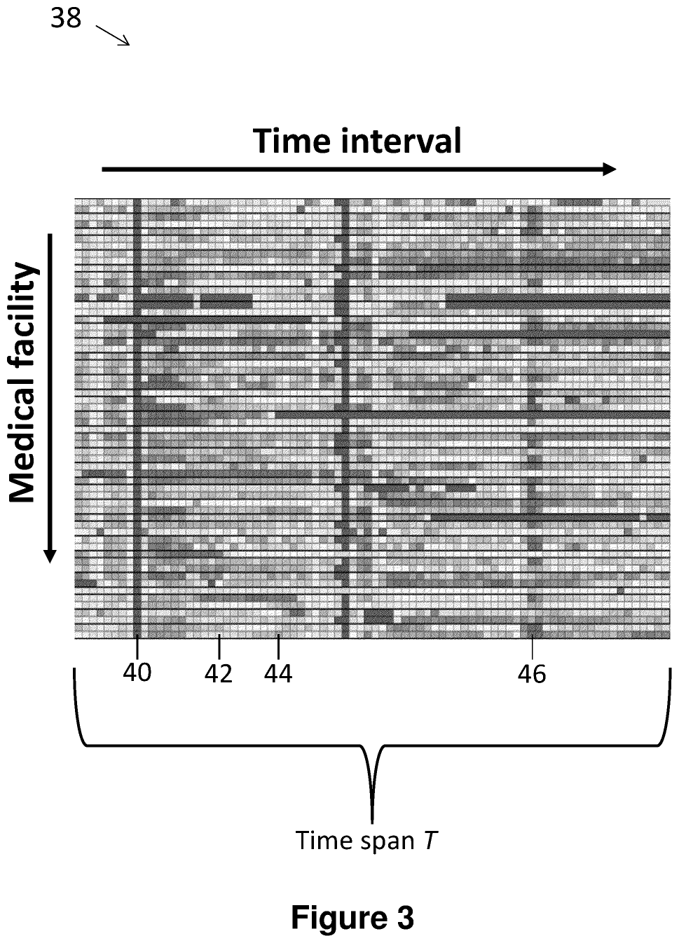

FIG. 3 shows an example of the heat map 38. The illustrative heat map 38 employs a grid of blocks indexed (i, j) where index i=1, . . . , n indexes the n medical facilities, and corresponds to rows of the heat map (i.e., the medical facilities form the vertical or “y-axis”) and index j=1, . . . , k indexes k time-ordered time intervals t₁, . . . , tₖ spanning the time span T and corresponds to columns of the heat map (i.e. the time intervals for the horizontal or “x-axis”). The heat map illustrated in FIG. 3 employs different monochrome shades for patent drawing purposes; however, these shades typically correspond to different colors in the actually implemented heat map. For example, the shade C₀ 40 corresponding to zero counts is labeled, along with a shade 42 corresponding to green, a shade 44 corresponding to yellow, and a shade 46 corresponding to red. The color C₀ 40 indicates that no alerts have been received for the given site and time interval. A vertical band of one or more columns having color C₀ indicates that no alerts have been received for all sites for that given time interval. This is the case for the 9-th column in the illustrative heat map of FIG. 3, which is labeled with the reference number 40 corresponding to color C₀. Such a vertical band is readily visually discernible, and thus quickly informs the viewer that the time interval corresponding to the vertical band of color Co likely corresponds to a time interval over which no alerts were received, most likely due to a delay in transmitting the alerts over that time interval, or a loss of communication over that time interval. A horizontal strip predominantly of one or two colors corresponding to high (per-facility scaled) alert counts, for example a horizontal strip of dark orange to red if those colors represent the high end of the alerts scale, indicates that for the specific site repeatedly a high number of alerts is received, which may indicate that the site has some serious issues, which could deserve further attention. Again, the user is quickly informed by the horizontal orange/red band that this site is likely experiencing a serious problem calling for immediate investigation. In addition to giving the zero counts a special color, a separate color, e.g., a darker shade of red, could be dedicated to counts that exceed an appropriately defined limit, e.g., μ+κ·δ, for an appropriately chosen value of κ, e.g. 10.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by an electronic processor to perform an alert monitoring method, the alert monitoring method comprising:
receiving, at the electronic processor and via an electronic network, alerts generated by n medical databases servicing respective medical entities, each alert being timestamped with a receipt time when the alert was received at the electronic processor and each alert being associated to the medical database that generated the alert;
generating a grid representation of the alerts over a time span T, the grid representation comprising a grid of blocks indexed (i, j) where index i=1, . . . , n indexes the n medical facilities and index j=1, . . . , k indexes k time-ordered time intervals t₁, . . . , tₖ spanning the time span T where the time intervals t₁, . . . , tₖ are of equal time duration, wherein each block (i, j) of the grid has a value a_{i,j} indicative of a number of alerts received from medical database i over the time interval tⱼ;
generating a heat map representing the alerts over the time span T and having blocks corresponding to the blocks indexed (i, j) of the grid representation by mapping the values a_{i,j} of the respective blocks of the grid representation to colors using a color scale; and
displaying the heat map on a color display that is operatively connected with the electronic processor.

2. The non-transitory computer readable medium of claim 1, wherein the color scale includes a first color, a second color, and a third color, and the mapping comprises:
mapping to the first color values a_{i,j} that are indicative of a high number of alerts received from medical database i over the time interval tⱼ;
mapping to the second color values a_{i,j} that are indicative of an intermediate number of alerts received from medical database i over the time interval tⱼ; and
mapping to the third color values a_{i,j} that are indicative of a low number of alerts received from medical database i over the time interval tⱼ.

3. The non-transitory computer readable medium of claim 2, wherein the color scale further includes a fourth color, and the mapping comprises:
mapping to the fourth color values a_{i,j} that are equal to zero.

4. The non-transitory computer readable medium of claim 1, wherein the mapping comprises:
constructing n medical database-specific color scales C₁, . . . , Cₙ corresponding to the n medical databases, the color scale Cᵢ for corresponding medical database i being scaled based on the values a_{i,1}, . . . , a_{i,k} for the alerts received from the medical database i; and
mapping the values a_{i,1}, . . . , aₖ for each respective medical database i to colors of the database-specific color scale Cᵢ constructed for the medical database i.

5. The non-transitory computer readable medium of claim 4, wherein the constructing of each database-specific color scale Cᵢ includes scaling by a median absolute difference of the values a_{i,1}, . . . , a_{i,k} for the alerts received from the medical database i.

6. The non-transitory computer readable medium of claim 5, wherein the constructing of each database-specific color scale Cᵢ includes scaling by a median absolute difference median (|x−μ|) where μ is the median value of the set {a_{i,1}, . . . , a_{i,k}}.

7. The non-transitory computer readable medium of claim 1, wherein the n medical databases comprise n respective Picture Archiving and Communication System (PACS) databases.

8. The non-transitory computer readable medium of claim 1, wherein the alert monitoring method further comprises:
receiving a selection of a block (i, j) of the displayed heat map via a user-operable input device; and
displaying a list of the alerts received from the medical database indexed i over the time interval tⱼ.

9. An alert monitoring apparatus, comprising a server computer programmed to:
receive alerts generated by n medical databases servicing respective medical entities, each alert being timestamped with a receipt time when the alert was received at the electronic processor and each alert being associated to the medical database that generated the alert;
generate a grid representation of the alerts over a time span T, the grid representation comprising a grid of blocks indexed (i, j) where index i=1, . . . , n indexes the n medical facilities and index j=1, . . . , k indexes k time-ordered time intervals t₁, . . . , tₖ spanning the time span T where the time intervals t₁, . . . , tₖ are of equal time duration, wherein each block (i, j) of the grid has a value a_{i,j} indicative of a number of alerts received from medical database i over the time interval tⱼ, wherein the mapping comprises:
constructing n medical database-specific C₁, . . . , Cₙ corresponding to the n medical databases, the color scale Cᵢ for corresponding medical database i being scaled based on the values a_{i,1}, . . . , a_{i,k} for the alerts received from the medical database i; and
mapping the values a_{i,1}, . . . , aₖ for each respective medical database i to colors of the database-specific color scale Cᵢ constructed for the medical database i;
wherein the constructing of each database-specific color scale Cᵢ includes scaling by a median absolute difference of the values a_{i,1}, . . . , a_{i,k} for the alerts received from the medical database i;
generate a heat map representing the alerts over the time span T and having blocks corresponding to the blocks indexed (i, j) of the grid representation by mapping the values a_{i,j} of the respective blocks of the grid representation to colors using a color scale; and
display the heat map on a color display that is operatively connected with the electronic processor.

10. The apparatus of claim 9, wherein the color scale includes a first color, a second color, and a third color, and the mapping comprises:
mapping to the first color values a_{i,j} that are indicative of a high number of alerts received from medical database i over the time interval tⱼ;
mapping to the second color values a_{i,j} that are indicative of an intermediate number of alerts received from medical database i over the time interval tⱼ; and
mapping to the third color values a_{i,j} that are indicative of a low number of alerts received from medical database i over the time interval tⱼ.

11. The apparatus of claim 10, wherein the color scale further includes a fourth color, and the mapping comprises:
mapping to the fourth color values a_{i,j} that are equal to zero.

12. The apparatus of claim 9, wherein the constructing of each database-specific color scale Cᵢ includes scaling by a median absolute difference median (|x−μ|) where μ is the median value of the set {a_{i,1}, . . . , a_{i,k}}.

13. The apparatus of claim 9, wherein the n medical databases comprise n respective Picture Archiving and Communication System (PACS) databases.

14. The apparatus of claim 9, wherein the alert monitoring method further comprises:
receiving a selection of a block (i, j) of the displayed heat map via a user-operable input device; and
displaying a list of the alerts received from the medical database indexed i over the time interval tⱼ.

15. An alert monitoring method, comprising:
receiving, at an electronic processor and via an electronic network, alerts generated by n medical databases servicing respective medical entities, each alert being timestamped with a receipt time when the alert was received at the electronic processor and each alert being associated to the medical database that generated the alert;
generating a grid representation of the alerts over a time span T, the grid representation comprising a grid of blocks indexed where index i=1, . . . , n indexes the n medical facilities and index j=1, . . . , k indexes k time-ordered time intervals t₁, . . . , tₖ spanning the time span T where the time intervals t₁, . . . , tₖ are of equal time duration, wherein each block (i, j) of the grid has a value a_{i,j} indicative of a number of alerts received from medical database i over the time interval tⱼ, wherein the mapping comprises:
constructing n medical database-specific color scales C₁, . . . , Cₙ corresponding to the n medical databases, the color scale Cᵢ for corresponding medical database i being scaled based on the values a_{i,1}, . . . , a_{i,k} for the alerts received from the medical database i; and
mapping the values a_{i,1}, . . . , aₖ for each respective medical database i to colors of the database-specific color scale Cᵢ constructed for the medical database i;
wherein the constructing of each database-specific color scale Cᵢ includes scaling by a median absolute difference of the values a_{i,1}, . . . , a_{i,k} for the alerts received from the medical database i;
wherein the constructing of each database-specific color scale Cᵢ includes scaling by a median absolute difference median (|x−μ|) where μ is the median value of the set {a_{i,1}, . . . , a_{i,k}};
generating a heat map representing the alerts over the time span T and having blocks corresponding to the blocks indexed of the grid representation by mapping the values a_{i,j} of the respective blocks of the grid representation to colors using a color scale; and
displaying the heat map on a color display that is operatively connected with the electronic processor.

16. The method of claim 15, wherein the color scale includes a first color, a second color, and a third color, and the mapping comprises:
mapping to the first color values a_{i,j} that are indicative of a high number of alerts received from medical database i over the time interval tⱼ;
mapping to the second color values a_{i,j} that are indicative of an intermediate number of alerts received from medical database i over the time interval tⱼ; and
mapping to the third color values a_{i,j} that are indicative of a low number of alerts received from medical database i over the time interval tⱼ.

17. The method of claim 16, wherein the color scale further includes a fourth color, and the mapping comprises:
mapping to the fourth color values a_{i,j} that are equal to zero.

18. The method of claim 15, wherein the constructing of each database-specific color scale Cᵢ includes scaling by a median absolute difference of the values a_{i,1}, . . . , a_{i,k} for the alerts received from the medical database i.

19. The method of claim 15, wherein the alert monitoring method further comprises:
receiving a selection of a block (i, j) of the displayed heat map via a user-operable input device; and
displaying a list of the alerts received from the medical database indexed i over the time interval tⱼ.

20. The method of claim 15, wherein the alert monitoring method further comprises:
using the heat map to predict a failure or malfunction of one or more of the n medical databases.
